(19) United States
(12) Patent Application Publication
(10) Pub. No.: US 2021/0346437 A1
(43) Pub. Date: Nov. 11, 2021

(54) NOVEL TREATMENT OF DYSPHAGIA

(71) Applicant: Board of Trustees of Southern Illinois University, Springfield, IL (US)

(72) Inventor: Sandra Ettema, Mechanicsburg, IL (US)

(21) Appl. No.: 17/313,277

(22) Filed: May 6, 2021

Related U.S. Application Data
(60) Provisional application No. 63/021,571, filed on May 7, 2020.

(51) Int. Cl. A61K 35/50; A61K 9/00; A61P 1/00; A61M 29/02

(52) U.S. Cl. CPC A61K 35/50; A61K 9/0019; A61M 2210/105; A61M 29/02; A61P 1/00

(57) ABSTRACT

This disclosure provides methods for treating dysphagia in a patient in need of esophageal dilation of his/her esophageal stricture. The methods according to this disclosure comprise:
i. injecting circumferentially into the esophageal stricture area a sterile pharmaceutical composition comprising a pharmaceutically acceptable fluid carrier and tissue matrix allograft particles composed of human amnion and chorion membranes, wherein the amnion membrane is de-epithelized; and
ii. performing pneumatic dilation of the esophageal stricture with a balloon dilator.

The methods of this disclosure are particularly suitable for treating a complex and scarred cervical esophageal stricture, including reoccurring cervical esophageal strictures. The present methods may be particularly beneficial for cancer patients, including head and neck cancer patients, who were previously treated with external beam radiation and/or chemotherapy and/or a surgery for his/her cancer.

### CROSS-REFERENCE To RELATED APPLICATIONS

This patent application claims the benefit of priority from U.S. provisional patent application 63/021,571 filed May 7, 2020, the entire disclosure of which is herein expressly incorporated by reference in its entirety.

### TECHNICAL FIELD

This disclosure relates to the field of medical treatments and procedures, including methods that utilize a bioactive tissue matrix allograft composed of dehydrated human amnion and chorion membranes in esophageal dilation procedures.

### BACKGROUND

Cervical esophageal strictures are encountered by many head and neck cancer patients who have undergone radiation treatment and have difficulties swallowing solid foods and even liquids. These patients experience distress at mealtimes and often isolate themselves secondary to self-consciousness during eating because of gagging/choking, increased length of time for eating and the requirement of special food preparation. Some patients require a feeding tube as they are not able to maintain adequate nutrition orally. This leads to psychologic depression, anxiety and decreased quality of life (QOL).

Esophageal strictures are categorized as malignant or benign. Dilation for malignant esophageal strictures is performed as a supplementary procedure in addition to stent placement/other procedure¹. Benign esophageal strictures can be classified into two types: 1) Simple: Schatzki rings, esophageal webs, peptic injury, etc. which have a low recurrence rate and 2) Complex: caustic ingestion, radiation injury, anastomotic strictures, etc. which are more difficult to dilate and have higher recurrence rates¹.

Kochhar and Poornachandra (2010) describe simple strictures as focal, straight, and symmetrical with a diameter of >12 mm and amenable to treatment with balloon dilation and generally require 1-3 dilations to achieve symptomatic relief. Complex esophageal strictures are >2 cm in length, tortuous, and asymmetrical with a diameter of <12 mm and are more difficult to treat requiring 3-8 dilations and associated with higher recurrence rates¹. Cervical esophageal strictures occur frequently in head and neck cancer patients treated with radiation. Standard recommendations for management of complex strictures have not been developed, but treatments have included dilation with intralesional steroid injection and/or endoscopic incisional treatment, placement of various types of stents, use of bougies, and endoscopic surgery¹. The latter option being more invasive and carrying higher risk to patients.

Currently there is no universally accepted definition of recurrent or refractory esophageal stricture, but some publications describe it as an anatomic restriction resulting from cicatricial luminal compromise or due to fibrosis leading to clinical symptoms of dysphagia in the absence of any endoscopic evidence of inflammation¹. It has been reported that a refractory stricture is the inability to resolve the anatomic stricture up to a diameter of 14 mm over 5 sessions at 2 week intervals and a recurrent stricture is due to the inability to maintain a 14 mm diameter for 4 weeks once the 14 mm diameter is achieved⁷. There are no standard recommendations for the management of these strictures which have been treated with dilation and intralesional steroid injection^{8,9}, dilation with endoscopic incisional treatment¹⁰, placement of various types of stents¹¹⁻¹³, bougies¹⁴, and endoscopic surgery¹⁵. The latter option being more invasive and carrying a higher risk to the patients in comparison to dilation and injection of steroid.

Intralesional steroid injections for benign refractory esophageal strictures have been practiced for >40 years². Otolaryngologists frequently use steroids in combination with esophageal dilation. The mechanism of action of steroids has been thought to prevent cross-linking of collagen that leads to decreased occurrence in scar contracture and formation¹. There are discrepancies with this method as steroids have inconsistently been shown to help maintain the dilation size or perhaps lengthen the times between dilations.

Various studies have been completed using intralesional steroid injections in conjunction with dilation to determine if steroid injection is actually beneficial and results have been inconsistent. In a recent multicenter, double-blind trial of 60 patients with anastomotic strictures after esophagectomy who underwent either steroid or saline injection followed by dilation demonstrated no statistically significant decrease in frequency of repeat dilations or prolongation of the dysphagia-free period³. But another double-blind randomized study of 19 patients with complex strictures after esophagectomy who were placed in groups of dilation only or dilation with injection of steroid demonstrated a significant improvement or resolution of dysphagia in the steroid group⁴. Currently, it is a standard to inject steroid as there have not been any detrimental side effects with incorporating steroid injection with endoscopic esophageal balloon dilation.

Steroids often help more with people who have esophageal stricture due to causes such as reflux, prominent cricopharyngeus, and possibly anastomotic strictures, but not as much in those patients who have undergone radiation treatment. Patients who have been irradiated have actual scar formation in that region of their esophagus where the tissue does not have pliable muscle properties, making it more difficult to perform a dilation procedure.

Thus, there remains the need in the field for treatment procedures for esophageal strictures and in particular, complex esophageal strictures.

### SUMMARY

The present disclosure helps in addressing this need. In one aspect, the present disclosure provides methods for treating dysphagia in a patient in need of esophageal dilation of his/her esophageal stricture. The methods according to this disclosure comprise:
i. injecting circumferentially into the esophageal stricture area a sterile pharmaceutical composition comprising a pharmaceutically acceptable fluid carrier and tissue matrix allograft particles composed of human amnion and chorion membranes, wherein the amnion membrane is de-epithelized; and
ii. performing pneumatic dilation of the esophageal stricture with a balloon dilator.

In some preferred embodiments of the methods, from about 40 mg to about 160 mg, and more preferably about 100 mg, of the tissue matrix allograft particles may be injected in step i. Some preferred embodiments of the pharmaceutical composition injected in step i. may comprise sterile saline as the fluid carrier. In some preferred embodiments of the methods, one injection dosage for the pharmaceutical composition is about 0.5 cc to about 2.0 cc and it contains from about 40 mg to about 160 mg of the tissue matrix allograft particles. The methods may further comprise preparing the sterile pharmaceutical composition by mixing dry powder containing the tissue matrix allograft particles with the pharmaceutically acceptable fluid carrier.

The composition injected in step i. is available under tradename AMNIOFIX™ injectable from Mimedx Group, Inc., Kennesaw, Ga.

The methods of this disclosure are particularly suitable for treating a complex and scarred cervical esophageal stricture, including reoccurring cervical esophageal strictures. The present methods may be particularly beneficial for cancer patients, including head and neck cancer patients, who were previously treated with external beam radiation and/or chemotherapy and/or a surgery for his/her cancer.

In some embodiments of the methods, step ii. (performing pneumatic dilation) may comprise:
a) Visualizing the cervical esophageal stricture in the patient's esophagus;
b) Placing a deflated balloon dilator into the cervical esophageal stricture area;
c) Inflating the balloon dilator and causing the inflated balloon dilator to press against the cervical esophageal stricture, and thereby dilating the stricture;
d) Deflating the balloon dilator;
e) Repeating steps c) and d) as may be necessary; and
f) removing the balloon dilator.

The injection (step i) may be performed at least prior to step b), step c) and/or prior to the final repetition of step c). In some preferred embodiments, the injection is performed at least before the final repetition of step c).

In some embodiments, a diameter to which the balloon dilator is inflated in step c) may be sequentially increased with each subsequent repetition. In some preferred embodiments, the balloon can be inflated for at least 3 minutes. In some embodiments of the methods, step a) can be performed with a laryngoscope and a 0-degree telescope.

In yet another aspect, the present disclosure provides methods of treating a cervical esophageal stricture, comprising injecting circumferentially into the cervical esophageal stricture area a sterile pharmaceutical composition comprising a pharmaceutically acceptable fluid carrier and tissue matrix allograft particles composed of human amnion and chorion membranes, wherein the amnion membrane is de-epithelized.

### BRIEF DESCRIPTION OF THE DRAWINGS

FIG. 1 illustrates the pre-dilation status of the upper esophageal region of a previously irradiated head and neck cancer patient prior to the standard procedure consisting of serial balloon dilation and injection of Kenalog into the stricture in February 2018.

FIG. 2 illustrates the same patient's upper esophageal region eight weeks later (April 2018) prior to the standard procedure of serial balloon dilation with injection of Kenalog. The patient was only eating shakes and liquids immediately before this point in time and the previous procedure noted with FIG. 1.

FIG. 3 illustrates the same patient's pre-procedure status of the upper esophageal region another eight weeks later (June 2018) where the procedure changed to serial balloon dilation with injection of 40 mg AMNIOFIX™ (Mimedx Group, Inc., Kennesaw, Ga.).

FIG. 4 illustrates the same patient's pre-procedure status of the upper esophageal region eleven weeks later (August 2018) where serial balloon dilation with a second injection of 40 mg AMNIOFIX™ was administered. The procedure was delayed 3 weeks secondary to the patient tolerating more solid foods for a longer period of time. The pre-procedure starting dilation diameter also increased from the previous 12 mm starting dilation (February, April and June) to 18 mm dilation (August—after 1^{st} AMNIOFIX™ injection in June 2018). The patient was dilated at 18 mm and then at 19 mm.

FIG. 5 illustrates the same patient's pre-procedure status of the upper esophageal region sixteen weeks later (December 2018) where serial balloon dilation starting at 18 mm in diameter and serially dilating to 19 mm and then 20 mm was completed with a third injection of 40 mg of AMMOFIX™ administered.

### DETAILED DESCRIPTION

In one aspect, this disclosure provides dilation methods for treating esophageal strictures, including scarred and nonpliable cervical esophageal strictures. The present methods are particularly suitable for complex esophageal strictures which are difficult to treat by conventional methods. These strictures may require 3 to 8 dilations and at least some of them may also have a high recurrence rate.

In this disclosure, an “esophageal stricture” means an abnormal tightening or narrowing of the esophagus. Two types of strictures are known: simple and complex. Simple strictures are usually straight and substantially symmetrical, while complex strictures are longer and leave a narrower opening. Complex esophageal strictures include those which are more than about 2 cm in length, tortuous, and/or asymmetrical. In some complex esophageal strictures, an esophageal diameter may be decreased to less than 12 mm. The present methods are particularly suitable for treating complex strictures, including those strictures which are at least partially scarred.

Patients having an esophageal stricture and in particular a cervical esophageal stricture, and further including complex esophageal strictures, may benefit from the methods according to this disclosure. Such patients may include head and neck cancer patients who were treated with radiation, chemotherapy and/or surgery for their condition, but any other patient with an esophageal stricture may also benefit from this procedure. Other patients may include, but are not limited to, patients with inflammation in the esophagus, patients with esophageal cancer, radiation therapy patients, chemotherapy patients, patients whose surgery and/or a procedure in the esophagus led to development of scars causing a stricture, patients with certain infections, patients who accidently swallowed a chemical or an object causing a scar to their esophagus, and patients with traumatic injuries that resulted in at least one scar leading to an esophageal stricture.

Some patients with a cervical esophageal stricture may have difficulties in swallowing solid food (dysphagia) and may experience choking episodes. The present methods are particularly beneficial to patients with dysphagia whose condition can be helped at least partially by esophageal dilation such as pneumatic dilation.

The methods according to this disclosure are particularly beneficial to head and neck cancer patients who undergo radiation and often develop radiation-induced esophageal strictures that are difficult to treat due to intense fibrosis and scarring. Despite improvement shown with intralesional steroids^{1,4}, many head and neck cancer patients still require multiple dilations to maintain some type of oral intake and some require feeding tubes to maintain adequate nutrition.

Some preferred embodiments of this disclosure relate to a method for treating dysphagia in a patient in need of esophageal dilation of his/her esophageal stricture, the method comprising:
i. injecting circumferentially into the esophageal stricture area a sterile pharmaceutical composition comprising a pharmaceutically acceptable fluid carrier and tissue matrix allograft particles composed of human amnion and chorion membranes, wherein the amnion membrane is de-epithelized; and
ii. performing pneumatic dilation of the esophageal stricture with a balloon dilator.

Pneumatic dilation with a balloon dilator is conventionally used in order to re-open (dilate) esophageal strictures. These procedures aid patients who otherwise may have difficulties with swallowing solid foods. Certain esophageal strictures, especially cervical complex strictures, are known to re-occur, requiring repetition of the dilation procedure every few weeks. Many complex strictures are also scarred and non-pliable and they may be very difficult to dilate.

This inventor found that it is possible to improve significantly the outcome of dilation by injecting into the esophageal stricture area, including the cervical esophageal stricture area, a pharmaceutical composition comprising tissue matrix allograft particles composed of human amnion and chorion membranes, wherein the amnion membrane is de-epithelized. The composition suitable for conducting the present methods is available under trademark AMMOFIX™ injectable from Mimedx Group, Inc., Kennesaw, Ga.

AMMOFIX™ injectable contains tissue matrix allograft particles composed of human amnion and chorion membranes derived from human placenta, wherein the amnion membrane is de-epithelized. AMNIOFIX™ injectable contains matrix proteins, growth factors, and cytokines which aid in reducing scar tissue formation.

This composition can be obtained by various protocols used in obtaining the amnion and chorion membranes from a donated placenta, including the following protocols described in U.S. Pat. No. 10,105,398, the entire disclosure of which is herein incorporated by reference. In brief and in accordance with U.S. Pat. No. 10,105,398, donated human placentas are collected from consenting mothers during a childbirth by Cesarean section. The amnion membrane is separated from the chorion membrane. The amnion membrane is then de-epithelized. In some preferred embodiments, the intermediate (spongy) layer of the amnion membrane may be also removed. The amnion and chorion membranes then undergo chemical decontamination and dehydration, essentially as described in U.S. Pat. No. 10,105,398. The dehydrated membranes, which may be laminated together prior to dehydration or left as two separate membranes, are then micronized in order to produce injectable powder.

As described in U.S. Pat. No. 10,105,398, micronized compositions can be produced by grinding and/or shredding of the dehydrated amnion and chorion membranes. The particle size of the dehydrated amnion and chorion membranes may vary. Preferably, but not limiting, the particles may be less than 500 micrometers, less than 400 micrometers, less than 300 micrometers, from 25 micrometers to 300 micrometers, from 25 micrometers to 200 micrometers, or from 25 micrometers to 150 micrometers; and more preferably, from 150 microns to 350 microns. If a specified size for particles in the ground composition is needed, e.g., from 150 microns to 350 microns, these particles can be sorted by passing the ground composition through a sieve, e.g., a series of American Standard ASTM sieves as also described in detail in U.S. Pat. No. 10,105,398. These injectable dry compositions may further comprise excipients, e.g., a filler, a solubilizer and/or additional active comments, e.g., collagen.

Various pharmaceutically acceptable fluid carriers, which may also be referred to as a fluid excipient, can be used for reconstituting the injectable composition. These fluid carriers may include, but are not limited to, water, aqueous hyaluronic acid, saline, Ringer's solution, dextrose solution, Hank's solution, and/or a buffer. Suitable buffers include, but are not limited to, a phosphate buffer, bicarbonate buffer, or Tris buffer. In the methods according to this disclosure, one particularly preferred fluid carrier is saline.

In the methods according to this disclosure, the pharmaceutical composition which is commercially available as AMNIOFIX™ injectable and which comprises tissue matrix allograft particles composed of human de-epithelized amnion and human chorion membranes can be injected in any amount sufficient to: 1) improve the pliability of the stricture; and/or 2) prevent or delay at least partially the recurrence of the stricture.

In some preferred embodiments from about 40 mg to about 160 mg and more preferably from 100 mg to 160 mg of the tissue matrix allograft particles composed of human amnion and chorion membranes, wherein the amnion membrane is de-epithelized, can be used. The composition can be injected in any volume suitable for injection, e.g., from about 0.5 cc to about 2 cc, but smaller or larger volumes can be also used.

In particularly preferred embodiments, the present methods comprise injecting into the esophageal stricture area from about 40 mg to about 160 mg, and preferably, from about 100 mg to 160 mg of the AMNIOFIX™ injectable composition. The composition can be injected in any volume suitable for injection, e.g., from about 0.5 cc to about 2 cc.

In the present methods, the injection may take place before the pneumatic dilation of the esophageal stricture with a balloon dilator. Furthermore, if a dilation procedure includes several sequential balloon inflations, as described in more detail below, then the injection can be performed at least before the final inflation, as described in more detail below.

In some preferred embodiments, the present methods include performing pneumatic dilation of the esophageal stricture with a balloon dilator. Any balloon dilator conventionally used for esophageal dilation, can be also used in the present methods. Typically, pneumatic dilation in the present methods may include at least the following steps:
a) Visualizing the cervical esophageal stricture in the patient's esophagus;
b) Placing a deflated balloon dilator into the cervical esophageal stricture area;
c) Inflating the balloon dilator and causing the inflated balloon dilator to press against the cervical esophageal stricture, and thereby dilating the stricture;
d) Deflating the balloon dilator;
e) Repeating steps c) and d) as may be necessary; and
f) removing the balloon dilator.

The injection with the composition comprising tissue matrix allograft particles composed of human amnion and chorion membranes, wherein the amnion membrane is de-epithelized, can be performed at least prior to step b), step c) and/or prior to the final repetition of step c).

In certain embodiments of the methods according to this disclosure, a diameter to which the balloon dilator is inflated in step c) may be sequentially increased with at least some of the subsequent repetitions. Steps c) and d) can be repeated as may be necessary. A medical practitioner can make a determination whether repeating steps c) and d) is necessary, based on the outcome of the first dilation and by evaluating tissue deformation, tension and/or tearing in order to avoid unnecessary damage to the tissue.

It has been discovered that using at least one injection of the AIVINIOFIX™ composition in the present methods may improve the dilation outcome as well make it easier for a patient to swallow solid food. Additional improvements in comparison to conventional dilation procedures with a pneumatic balloon dilator in which no injection of the AMNIOFIX™ composition was used, include, but are not limited to: 1) improvement in the pliability of the esophageal tissue observed by the investigator; 2) increased tolerance of more solid foods prior to the patient's next dilation; 3) an increased time between dilations; and 4) reduction in the need for further procedures. All these factors may lead to improvement in safety for the patient and less complications, decreased healthcare costs, and improved quality of life (QOL) for the patient as they are able to tolerate more solid foods by mouth with less choking/gagging.

The AMNIOFIX™ composition is a bioactive tissue matrix allograft composed of dehydrated human amnion/chorion membrane that preserves and contains multiple extracellular matrix proteins, growth factors, and cytokines that help regulate inflammation, reduce scar tissue formation, and enhance surgical wound healing⁵. The AMNIOFIX™ composition is a durable, bioactive tissue matrix allograft composed of human amnion/chorion membrane processed through the proprietary PURION™ Process that protects the scaffold during processing which leaves an intact collagen matrix of proteins, growth factors, and cytokines which aid in reducing scar tissue formation⁶. The AMNIOFIX™ composition is intended for homologous use in chronic wound healing, reduction of scar formation, and modulating inflammation and was recently FDA approved for use in patients with osteoarthritis of the knee as it reduces pain and improves function⁶.

However, the methods according to this disclosure use the AMNIOFIX™ composition in conjunction with a dilation procedure in which the AMNIOFIX™ composition is injected into the scarred, nonpliable cervical esophageal strictures of a patient, for example, an irradiated head and neck cancer patient who has developed complex/refractory esophageal strictures and dysphagia. It is unexpected that the composition was beneficial in improving an outcome of a dilation procedure and also helped in delaying the recurrence of the stricture.

### EXAMPLE 1

A case series was performed that demonstrates injection of the AMNIOFIX™ composition into scarred cervical esophageal strictures of irradiated head and neck cancer patients improving the pliability of the tissue of this region leading to improved swallowing function as well as increased time between needed dilations.

Eight head and neck cancer patients with a history of radiation treatment resulting in complex cervical esophageal strictures with complaints of difficulty swallowing solids and liquids and narrowing of the upper esophageal region on video oropharyngeal swallow studies (VOSS) are presented.

Each patient underwent serial balloon dilation of cervical esophageal stricture with injection of 40, 80 (2×40), and/or 100 mg of the AMNIOFIX™ composition at least once and up to six times over a course of 18 months. Seven patients were male, one female, and their ages ranged between 44 and 72 years with a mean of 58. These patients required repeated dilation from 8-16 weeks consistently.

Serial cervical esophageal balloon dilations were performed in conjunction with “off-label” injection of the AMNIOFIX™ composition into the scarred region of esophageal strictures with all 8 patients.

Specific operative standard procedures performed included the following. A rigid 10×14 mm esophagoscope was attempted to be placed initially and was used for assessment of the stricture and determination of the initial size of the balloon dilator. Next, a laryngoscope was placed for adequate visualization of the cervical esophageal stricture and using a 0-degree telescope the stricture will be dilated sequentially inflating the balloon dilators at the appropriate atmospheres at each diameter for approximately 3 minutes. Prior to the final dilation based on patient tissue deformation, tension and/or tearing, approximately 40 mg, 80 (2×40) mg or 100 mg of AMNIOFIX™ composition reconstituted in 1.5 cc of sterile saline was injected circumferentially into the strictured area using the 0-degree telescope for visualization. The final dilation was then completed as per the above protocol.

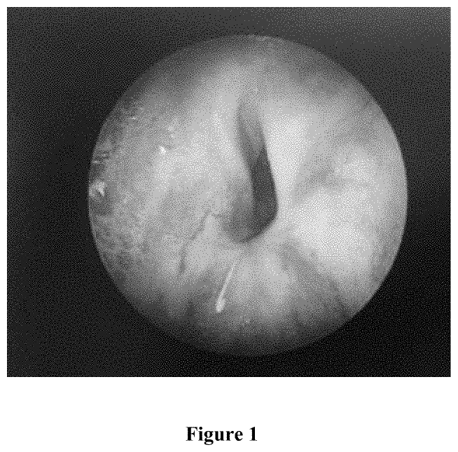

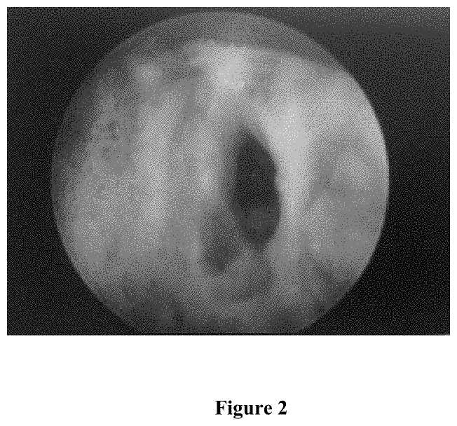

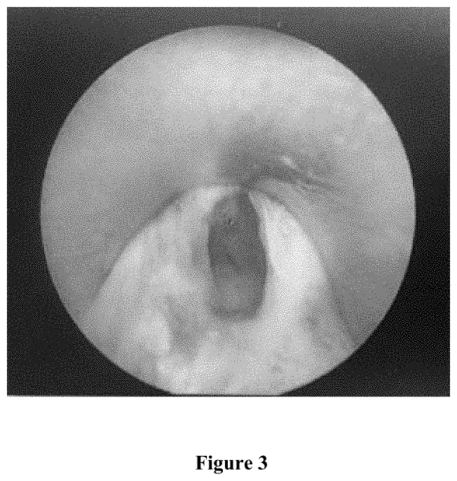

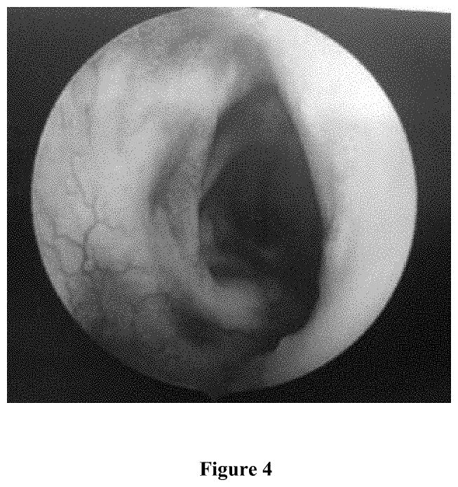

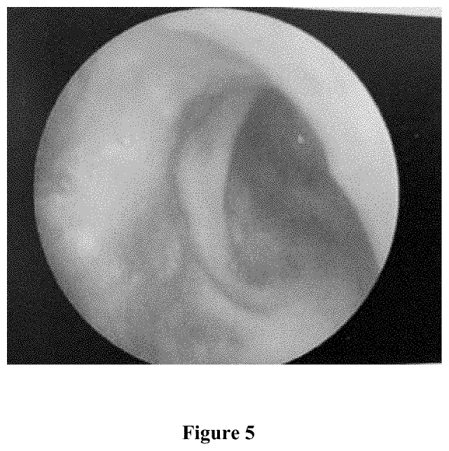

One patient demonstrated improvement of dysphagia and after treatment, demonstrated an ability to swallow diced foods as opposed to pureed foods or liquids with continued dilations at 12-16 week intervals. Four patients demonstrated improvement in dysphagia not requiring further dilations after 1-3 procedures. Two patients demonstrated improvement of dysphagia as well as an increased number of weeks/months between dilations (see FIGS. 1-5). Four patients demonstrated subjective increased pliability and softening of the region of the cervical esophageal stricture, requiring a larger balloon diameter for dilation at subsequent procedures. Each patient was followed and demonstrated an improvement in their ability to swallow more solid foods and/or experienced increased time intervals between esophageal dilations, requiring less frequent procedures in the operating room. All patients subjectively stated and are extremely satisfied with the results after their procedures with injection of the AMNIOFIX™ composition.

The injection of AMNIOFIX™ composition was performed at one of three dosages which include 40 mg, 80 mg and 100 mg. The patient depicted in FIGS. 1-5 illustrates the use of a 40 mg AMNIOFIX™ injection each time.

After this patient, the other patients were treated with 80 mg and 100 mg treatment dosages secondary to investigator assessment of the scar severity and/or hospital availability of the allograft.

In all procedures, the active amount of the AMNIOFIX™ composition, which is provided in a dry cell format, is reconstituted with 1-2 cc of sterile injectable saline. The amount of sterile injectable saline used depended on the area needed for treatment. The larger the area, the greater the amount of sterile injectable saline used. The most typical amount used is 1.5 cc of sterile injectable saline. The drug was injected just before the last dilation of the procedure and dilations occurred from 6 weeks to 6 months or longer.

In these treatment methods, serial cervical esophageal balloon dilations via direct laryngoscopy with a 0-degree telescope for visualization was performed and an orotracheal injector was employed for the injection of the AMNIOFIX™ composition into the scarred region of esophageal strictures. One patient demonstrated improvement of dysphagia and is able to swallow diced foods vs. pureed or liquids with continued dilations at 12-16 week intervals. Six patients demonstrated improvement in dysphagia not requiring further dilations after 1-3 procedures. Four patients demonstrated improvement of dysphagia as well as increased number of weeks/months between dilations. Five patients demonstrated subjective increased pliability and softening of the cervical esophageal stricture and requiring a larger balloon diameter for dilation at subsequent procedures. Each patient continues to be followed and has demonstrated improvement in ability to swallow more solid foods and/or increased time between esophageal dilations requiring less frequent procedures in the operating room. The data demonstrating potential subjective improvement in swallowing are summarized in Table I.

TABLE I
Data Demonstrating Subjective Improvement in Swallowing

| | # Dilations Prior | | # Dilations With | Increased Tissue Pliability- |
|---|---|---|---|---|
| | AmnioFix/# weeks | | AmnioFix/# weeks | Larger Starting-Ending |
| Patient | Between Procedures | | Between Each Procedure† | Diameter Comparisons‡ |
| A | >9/Average 16-20 weeks | | 2/16 weeks and holding | 8-18 mm/9-18 mm |
| B | N/A | | 2/14 weeks and holding | 12-18 mm/13.5-20 mm |
| C | 1/22 | weeks | 3/14 weeks; 52 weeks and | (15-20 mm for all) |
| | | | holding | |
| D | 3/8 | weeks | 7/every 12-16 weeks | (12-20 mm for all) |
| E | 1/25 | weeks | 3/12 weeks; 35 weeks and | 15-18 mm/18-20 mm |
| | | | holding | |
| F | 3/8 | weeks | 3/9 weeks; 9 weeks and | 10-18 mm/10-20 mm |
| | | | holding | |
| G | 9/14-16 | weeks | 1/holding (>43 weeks) | N/A - has not required |
| | | | | another dilation |
| H | 6/6-8 | weeks | 5/11 weeks; 16 weeks; 19 | 12-18 mm/18-20 mm |

weeks; 32 weeks and
holding

†Holding means patient has not returned for another dilation since last injection of AmnioFix;
‡First AMNIOFIX ™ Injection/Last AMNIOFIX ™ Injection

As demonstrated in this case series, the AMNIOFIX™ composition has been injected into the complex cervical esophageal strictures of irradiated head and neck cancer patients, without complications, and demonstrated improvement in dysphagia of all patients. With this treatment, patients experience a toleration of more solid food consistencies for longer periods of time between dilations. Less frequent dilations lead to a decreased number of patient procedures which improves safety, decreases healthcare costs, and increases quality of life for these patients.

### REFERENCES

1. Kochhar R, Poornachandra K S. Intralesional steroid injection therapy in the management of resistant gastrointestinal strictures. World J Gastrointest Endosc. 2010 Feb. 16; 2(2):61-8. doi: 10.4253/wjge.v2.i2.61.
2. Ramage J I Jr, Rumalla A, Baron T H, Pochron N L, Zinsmeister A R, Murray J A, Norton I D, Diehl N, Romero Y. A prospective, randomized, double-blind, placebo-controlled trial of endoscopic steroid injection therapy for recalcitrant esophageal peptic strictures. Am J Gastroenterol. 2005; 100:2419-2425.
3. Hirdes M M, van Hooft J E, Koornstra J J, Timmer R, Leenders M, Weersma R K, Weusten B L, van Hillegersberg R, van Berge Henegouwen M I, Plukker J T, Wiezer R, Bergman, J G, Bleggaar F P, Fockens P, and Siersema P D. Endoscopic corticosteroid injections do not reduce dysphagia after endoscopic dilation therapy in patients with benign Esophagogastric anastomotic strictures. Clin Gastroenterol Hepatol. 2013 July:11(7):795-801. doi: 10.1016/j.cgh.2013.01.016.
4. Pereira-Lima J C, Lemos Bonotto M, Hahn G D, Watte G, Lopes C V, dos Santos C E, Teixeira C R. A prospective randomized trial of intralesional triamcinolone injections after endoscopic dilation for complex Esophagogastric anastomotic strictures. Surg Endosc. 2015 May; 29(5):1156-60. doi: 10.1007/s00464-014-3781-6.
5. Koob T J, Lim J J, Massee M, Zabek N, Rennert R, Gurtner G, Li W W. Angiogenic properties of dehydrated human amnion/chorion allografts: therapeutic potential for soft tissue repair and regeneration. Vasc Cell. 2014 May 1; 6:10.
6. MiMedx Group, Inc. 2016. http://www.mimedx.com
7. Kochman M L, McClave S A, Boyce H W. The refractory and the recurrent esophageal stricture: a definition. Gastrointest Endosc. 2005; 62:474-475.
8. Zein N N, Greseth J M, Perrault J. Endoscopic intralesional steroid injections in the management of refractory esophageal strictures. Gastrointest Endosc. 1995; 41:596-598
9. Kochhar R, Makharia G K. Usefulness of intralesional triamcinolone in treatment of benign esophageal strictures. Gastrointest Endosc. 2002; 56:829-834.
10. DiSario J A, Pedersen P J, Bichiey-Canoutas C, Alder S C, Fang J C. Incision of recurrent distal esophageal (Schatzki) ring after dilation. Gastrointest Endosc. 2002; 56:244-248.
11. Fiorini A, Fleischer D, Valero J, Israeli E, Wengrower D, Goldin E. Self-expandable metal coil stents in the treatment of benign esophageal strictures refractory to conventional therapy: a case series. Gastrointest Endosc. 2000; 52:259-262.
12. Radecke K, Gerken G, Treichel U. Impact of a self-expanding, plastic esophageal stent on various esophageal stenoses, fistulas, and leakages: a single-center experience in 39 patients. Gastrointest Endosc. 2005; 61:812-818.
13. Fry S W, Fleischer D E. Management of a refractory benign esophageal stricture with a new biodegradable stent. Gastrointest Endosc. 1997; 45:179-182.
14. Kim C H, Groskreutz J L, Gehrking S J. Recurrent benign esophageal strictures treated with self-bougienage: report of seven cases. Mayo Clin Proc. 1990; 65:799-803.
15. Beilstein M C, Kochman M L. Endoscopic incision of a refractory esophageal stricture: novel management with an endoscopic scissors. Gastrointest Endosc. 2005; 61:623-625.

What is claimed is:

1. A method for treating dysphagia in a patient in need of esophageal dilation of his/her esophageal stricture, the method comprising:
i. injecting circumferentially into the esophageal stricture area a sterile pharmaceutical composition comprising a pharmaceutically acceptable fluid carrier and tissue matrix allograft particles composed of human amnion and chorion membranes, wherein the amnion membrane is de-epithelized; and
ii. performing pneumatic dilation of the esophageal stricture with a balloon dilator.

2. The method of claim 1, wherein step ii. comprises:
a) Visualizing the cervical esophageal stricture in the patient's esophagus;
b) Placing a deflated balloon dilator into the cervical esophageal stricture area;
c) Inflating the balloon dilator and causing the inflated balloon dilator to press against the cervical esophageal stricture, and thereby dilating the stricture;
d) Deflating the balloon dilator;
e) Repeating steps c) and d) as may be necessary; and
f) removing the balloon dilator.

3. The method claim 2, wherein step i. is performed at least prior to step b), step c) and/or prior to the final repetition of step c).

4. The method of claim 2, wherein a diameter to which the balloon dilator is inflated in step c) is sequentially increased with each subsequent repetition.

5. The method of claim 2, wherein step i. is performed at least before the final repetition of step c).

6. The method of claim 1, wherein from about 40 mg to about 160 mg of the tissue matrix allograft particles are injected in step i.

7. The method of claim 1, wherein about 100 mg of the tissue matrix allograft particles are injected in step i.

8. The method of claim 1, wherein the fluid carrier contains sterile saline.

9. The method of claim 1, wherein the balloon dilator is kept inflated for about 3 minutes.

10. The method of claim 1, wherein the patient is a cancer patient and the patient was previously treated with external beam radiation and/or chemotherapy and/or a surgery for his/her cancer.

11. The method of claim 2, wherein step a) is performed with a laryngoscope and a 0-degree telescope.

12. The method of claim 1, wherein the esophageal stricture is a cervical esophageal stricture and it is complex and scarred.

13. The method of claim 1, wherein the esophageal stricture is a reoccurring cervical esophageal stricture.

14. The method of claim 1, wherein the patient is a head and neck cancer patient.

15. The method of claim 1, wherein the method further comprises preparing the sterile pharmaceutical composition by mixing dry powder containing the tissue matrix allograft particles with the pharmaceutically acceptable fluid carrier.

16. The method of claim 1, wherein one injection dosage for the pharmaceutical composition is from about 0.5 cc to about 2.0 cc and it contains from about 40 mg to about 160 mg of the tissue matrix allograft particles.

17. A method of treating a cervical esophageal stricture, the method comprising injecting circumferentially into the cervical esophageal stricture area a sterile pharmaceutical composition comprising a pharmaceutically acceptable fluid carrier and tissue matrix allograft particles composed of human amnion and chorion membranes, wherein the amnion membrane is de-epithelized.
